(19) United States
(12) Patent Application Publication
(10) Pub. No.: US 2025/0195166 A1
(43) Pub. Date: Jun. 19, 2025

(54) DYNAMIC ADJUSTMENT OF SYSTEM FEATURES, CONTROL, AND DATA LOGGING OF SURGICAL ROBOTIC SYSTEMS

(71) Applicants: Covidien LP, Mansfield, MA (US); Digital Surgery Limited, London (GB)

(72) Inventors: William J. Peine, Ashland, MA (US); Danail V. Stoyanov, London (GB)

(21) Appl. No.: 18/846,741

(22) PCT Filed: Apr. 19, 2023
(86) PCT No.: PCT/EP2023/060191

§ 371 Date: Sep. 13, 2024

Related U.S. Application Data
(60) Provisional application No. 63/332,730, filed on Apr. 20, 2022.

(51) Int. Cl. A61B 34/00; A61B 34/35

(52) U.S. Cl. CPC A61B 34/77; A61B 34/35; A61B 34/74

(57) ABSTRACT

A surgical robotic system includes a robotic arm, a surgical console, and a computer. The robotic arm includes a surgical instrument and the surgical console includes a handle communicatively coupled to the robotic arm or the surgical instrument. The computer is configured to determine a phase or a task of a surgical procedure based on at least one of sensor data or a user command to perform the task, change a range of motion of one or more joints of the robotic arm or the surgical instrument based on the phase or the task of the surgical procedure, change a speed limit of the robotic arm based on the phase or the task of the surgical procedure, and change a rate of wireless transmission of data based on the phase or the task of the surgical procedure.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm.

For any electro-mechanical system, the functionality, reliability and life expectancy of the system depends on a variety of factors, such as the temperature at which it is operated. Power consumption of surgical robotic systems causes heat generation which, in the long-term, reduces the life expectancy of the system, and in the short-term, increases the risk of system-failure during operation. Additionally, the excessive usage of bandwidth during operation of a remotely operated surgical robotic system poses a boundary to tele-surgical implementations. Accordingly, there is a need to better utilize the limited bandwidth and storage space for data logging of procedure-relevant information.

### SUMMARY

According to one embodiment of the present disclosure, a surgical robotic system is disclosed. The surgical robotic system includes a robotic arm, a surgical console, and a computer. The robotic arm includes a surgical instrument and the surgical console includes a handle communicatively coupled to the robotic arm or the surgical instrument. The computer is configured to determine a phase or a task of a surgical procedure based on at least one of sensor data or a user command to perform the task, change a range of motion of one or more joints of the robotic arm or the surgical instrument based on the phase or the task of the surgical procedure, change a speed limit of the robotic arm based on the phase or the task of the surgical procedure, and change a rate of wireless transmission of data based on the phase or the task of the surgical procedure.

In an aspect, the computer is further configured to change a speed limit of the robotic arm based on the phase or the task of the surgical procedure by increasing the speed limit of the robotic arm when the task is determined to be initial dissection.

In an aspect, the computer is further configured to change a speed limit of the robotic arm based on the phase or the task of the surgical procedure by decreasing the speed limit when the task is determined to be a safety critical task.

In an aspect, the computer is further configured to change a range of motion of one or more joints of the robotic arm based on the phase or the task of the surgical procedure by changing the motion scaling between the handle and the robotic arm.

In an aspect, the computer is further configured to change a range of motion of the surgical instrument based on the phase or the task of the surgical procedure by reducing the range of motion of the surgical instrument when the task is to be performed in a predefined area.

In an aspect, the computer is further configured to change an input mapping between the handle and the robotic arm and the surgical instrument based on the phase or the task of the surgical procedure.

In an aspect, the computer is further configured to change the input mapping by amplifying rotation commands of the handle, remapping angles of the handle to change a start position of the handle, or changing control gains in the robotic arm.

In an aspect, the computer is further configured to cause a display device to overlay measurement scaling over surgical images, display contrast media, display visual enhancements, or display at least one pre-operative image matching a current surgical view based on the phase or the task of the surgical procedure.

In an aspect, the computer is further configured to change a rate of wireless transmission of data based on the phase or the task of the surgical procedure by increasing the rate of wireless transmission of data during dissection and suturing and reducing the rate of wireless transmission of data when a user is disengaged or when an instrument exchange is being performed.

In an aspect, the computer is further configured to record arm torques in response to a determination by the computer that the task is retraction of the robotic arm or retraction of the surgical instrument, record grasping force of the surgical instrument in response to a determination by the computer that the task is fine manipulation, and stop record signals in response to a determination by the computer that a user is disengaged from the surgical console.

In an aspect, the computer is further configured to change a data recording rate based on the phase of the surgical procedure and a speed of the surgical instrument by increasing the data recording rate when the speed of the surgical instrument is increased.

In an aspect, the computer is further configured to determine if the phase or task is outside of an expected metrics range, and record data with high fidelity information when it is determined that the phase or task is outside of the expected metrics range.

According to another embodiment of the present disclosure, a surgical robotic system is disclosed. The surgical robotic system includes a surgical console and a computer. The surgical console includes a handle communicatively coupled to at least one of a robotic arm or a surgical instrument. The computer is configured to determine a phase or a task of a surgical procedure based on at least one of sensor data or a user command to perform the task, determine whether the phase or task is to be performed in a predefined surgical area, reduce a range of motion of the robotic arm or the surgical instrument when the task is to be performed in the predefined surgical area, determine whether the phase or task is a safety critical task, and decrease a speed limit of the robotic arm when the task is determined to be a safety critical task.

In an aspect, the computer is further configured to determine if the phase or task is outside of an expected metrics range and record data with high fidelity information when it is determined that the phase or task is outside of the expected metrics range.

In an aspect, the computer is further configured to determine whether the task is retraction and record arm torque data when it is determined that the task is retraction.

In an aspect, the computer is further configured to determine whether the task is fine manipulation and record grasping force when it is determined that the task is fine manipulation.

According to another embodiment of the present disclosure, a method for dynamic adjustment of a surgical robotic system is disclosed. The method includes determining a phase or a task of a surgical procedure based on at least one of sensor data or a user command to perform the task, determining if the phase or task is outside of an expected metrics range, recording data with high fidelity information when it is determined that the phase or task is outside of the expected metrics range, determining whether the phase or task is to be performed in a predefined surgical area, and reducing a range of motion of the robotic arm or the surgical instrument when the task is to be performed in the predefined surgical area.

In an aspect, the method further includes recording arm torques when it is determined that the task is retraction of the robotic arm or retraction of the surgical instrument, recording grasping force of the surgical instrument when it is determined that the task is fine manipulation, and stopping the recording of signals when it is determined that a user is not engaged with the system.

In an aspect, the method further includes adjusting a data recording or wireless transmission rate when a speed of the surgical instrument is modified.

In an aspect, the method further includes changing a rate of wireless transmission of data based on the phase or the task of the surgical procedure by increasing the rate of wireless transmission of data during dissection and suturing and reducing the rate of wireless transmission of data when a user is disengaged or when an instrument exchange is being performed.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure; and

FIG. 5 is a flowchart illustration a method for dynamically adjusting a surgical robotic system.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a user console, a control tower, and one or more mobile carts having a surgical robotic arm coupled to a setup arm. The user console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

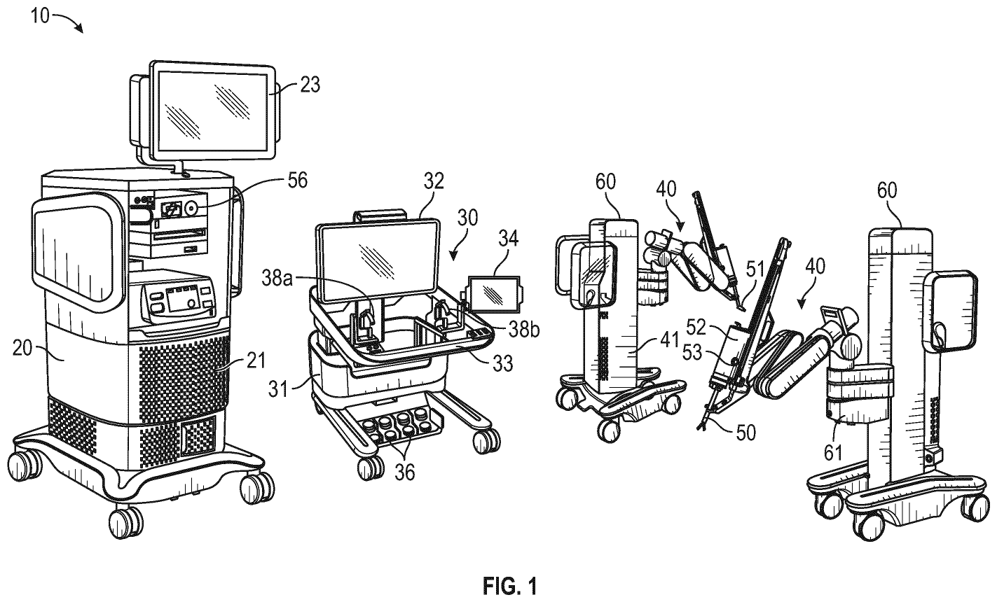

With reference to FIG. 1, a surgical robotic system 10 includes a control tower 20, which is connected to all of the components of the surgical robotic system 10 including a user console 30 and one or more movable carts 60. Each of the movable carts 60 includes a robotic arm 40 having a surgical instrument 50 removably coupled thereto. The robotic arms 40 also couple to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The user console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The user console 30 also includes a plurality of user interface devices, such as foot pedals 36 and a pair of handle controllers 38a and 38b which are used by a user to remotely control robotic arms 40. The user console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the user console 30 and one or more robotic arms 40. In particular, the control tower 20 is configured to control the robotic arms 40, such as to move the robotic arms 40 and the corresponding surgical instrument 50, based on a set of programmable instructions and/or input commands from the user console 30, in such a way that robotic arms 40 and the surgical instrument 50 execute a desired movement sequence in response to input from the foot pedals 36 and the handle controllers 38a and 38b.

Each of the control tower 20, the user console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

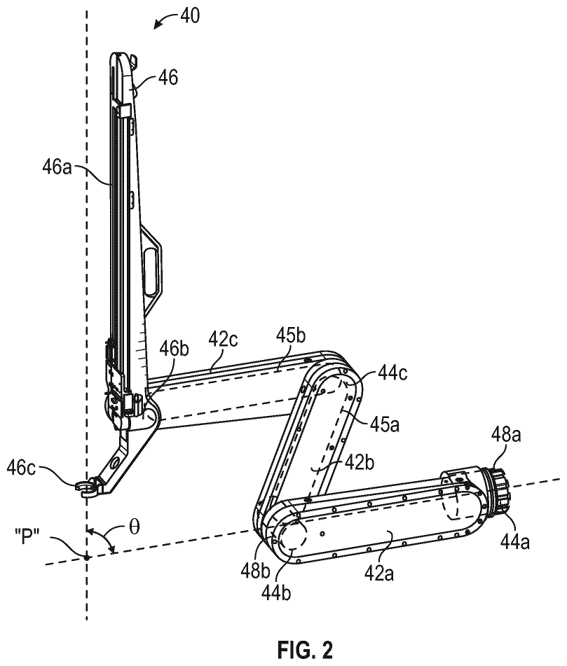

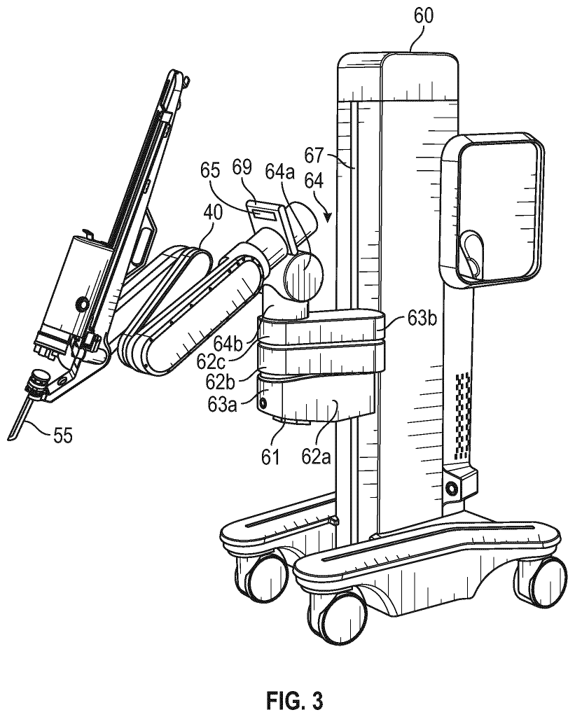

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

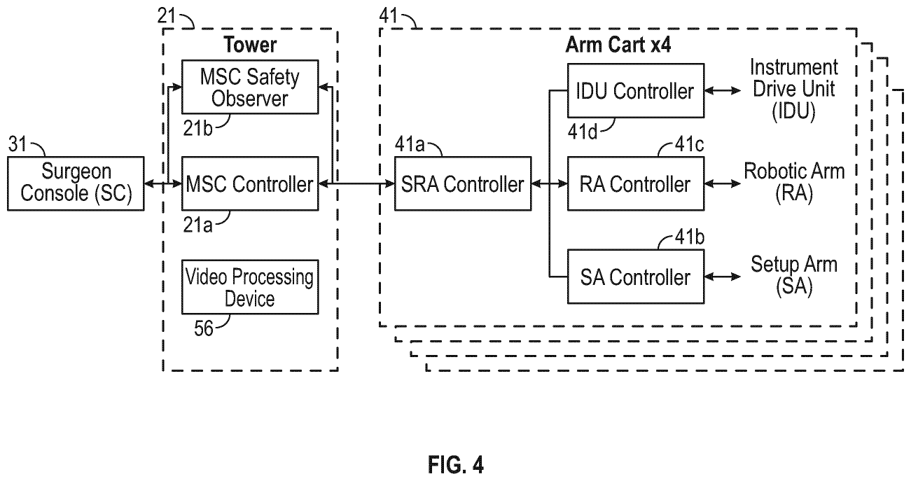

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the user console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the user console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or may be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the user console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The present disclosure provides a control algorithm, which may be embodied as software instructions executed by a controller, e.g., the controller 21a or any other suitable controller of the system 10. The control algorithm detects the current temporal phase, step, or commanded task of the surgical procedure in real-time and automatically adjusts the control and/or data logging functions of one or more components of the system 10 based on the detected phase, step, or commanded task. This is advantageous because it allows the system 10 to appropriately and dynamically change behavior based on the current surgical task or process and better utilize the limited computational and/or communication bandwidth and storage space for data logging of procedure-relevant and phase-relevant information.

The control algorithm detects the phase, step, or commanded task based on one or more sensors coupled to one or more components of the system 10, one or more sensors placed within the surgical setting (e.g., laparoscopic video camera), commands received from a user (e.g., commands received via any input devices such as handle controllers 38a and 38b or user interfaces), and/or inputs to and outputs form one or more other controllers of system 10. Based on the sensed data and/or received commands, the control algorithm determines the phase of the procedure, for example, initial surgical room preparation, robotic arm positioning, surgical instrument attachment, initial dissection, fine manipulation/dissection, grasping, suturing, etc. and may also categorize the phase or task, for example, as a safety-critical task. Depending on the procedure type, the control algorithm may also determine the next phase or task that follows the phase or task and perform a function based on the next phase or task. That is, the control algorithm of the system 10 could pre-emptively adjust information displays for the operating room team to optimize preparation of the next phase (e.g., prepare relevant instrumentation, notify relevant users that will be required in the next step, etc.).

In an aspect, the control algorithm changes the range of motion of one or more joints 48a, 48b, 48c, of the robotic arms 40 based on the detected task. In certain situations, the movable carts 60 may be placed closer together with a smaller range of motion to avoid collisions, with the range of motion shifting in real-time during the procedure depending on the surgical task and location of the current operative site. The joint limits may be setup as hard boundaries or soft boundaries that decrease speed limits or adjust the limit of other arm joints as the user moves away from the normal working range. The control algorithm may also change the speed limit of the robotic arms 40 or components of the surgical instrument 50 based on the surgical task. In certain embodiments, the control algorithm increases the speed limit of the robotic arms 40 during initial dissection and decreases the speed limits for safety-critical tasks or small scale tasks (e.g., fine dissection, suturing, etc.). In such an embodiment, the control algorithm may detect that the task is an initial dissection based on the elapsed time from the start of the procedure, based on the specific surgical instrument 50 being controlled, based on an explicit input by the user indicating that the action being performed is initial dissection, based on motion sensors, based on the position of the robotic arm 40 or surgical instrument 50 relative to the patient, based on one or more other sensors within the operating room, or any other such means or combinations thereof. Once the control algorithm determines that the task is an initial dissection, the control algorithm sets the speed limit of the robotic arm 40 and/or surgical instrument 50 accordingly. In one such configuration, the control algorithm dynamically reduces the speed limit of the robotic arm 40 and/or surgical instrument 50 as the surgical instrument 50 approaches the patient, that is, based on the distance of the surgical instrument 50 relative to the patient.

The control algorithm may also dynamically modify the motion scaling between the handle controllers 38a and 38b and the surgical instrument 50 based on the detected phase or task, for example, with smaller scaling for tasks that require large sweeping motions (e.g., moving the bowel around) and higher scaling for tasks that require small careful motions (e.g., fine dissection or suturing). In an aspect, the control algorithm may scale to accommodate for patient-specific information (e.g., accounting from BMI). The control algorithm may alternate mapping between the handle controllers 38a and 38b and the tip of the surgical instrument 50 when suturing to allow easier actuation of the motions required (amplified rotations, remapping of angles so that more comfortable hand positions are used, etc.). Additionally, or alternatively, the control algorithm may change the PD control gains in the joints 48a, 48b, 48c of the robotic arms 40 to improve tracking accuracy while reducing speed limits to avoid instability, changing the velocity compensation levels to improve the dynamic response of the system 10 or optimally compensate for backlash.

In educational or training configurations, when the system 10 is being used by a user for the first time, the control algorithm may alter the allowed range of motion of the surgical instrument 50 inside the patient based on the current surgical phase, step, or task. This improves safety while users are still learning the system 10 and performing initial cases. Experts may use this feature to provide safety zones so they can operate faster with less worry about accidental injury. In such a configuration, the control algorithm may create safety zones around a patient, or a user may designate safety zones around the patient, where the control algorithm will reduce the range of motion and/or speed of the robotic arm 40 and/or surgical instrument 50, when the surgical instrument 50 approaches or enters the safety zone.

The control algorithm may also cause the system 10 to initiate certain applications, modify graphical user interfaces and items displayed, and/or display pop-ups based on the detected phase or task. For example, a so-called follow-me mode, where camera angles are adjusted to follow movements of another surgical instrument, or other camera control schemes could automatically change depending on phase or task to optimize the surgical field of view or apply some specific presets on distance between the camera 51 and the site. The control algorithm may cause measurement applications to initiate at a specific point in the surgery to determine the size of a structure or record an image with a virtual ruler overlaid on the screen. This can link to specific organ or tissue identification capabilities or to instrument behaviors. Visualization enhancements (e.g., contrast media, virtual constraints, pre-operative imaging, etc.) may also be automatically initiated when the control algorithm detects that the user reaches or approaches an appropriate corresponding phase of the procedure. Alternatively, an icon may pop-up on the display (e.g., display 32 of surgical console 30) for the user to activate or deactivate the visualization enhancement features, as desired, based on the current temporal surgical phase or task. In one particular aspect, for example, pre-operative imaging may be automatically displayed on the display 32 of the surgical console 30 or other operating room team interface, such as display 23 of control tower 20, when the user reaches or approaches a specific step in the surgical procedure. These pre-operative images may be positioned/orientated in an appropriate relative orientation to match them to the surgical view.

The control algorithm may additionally, or alternatively, dynamically select or change the type, frequency, or amount and rate of data logging based on the surgical phase or task detected. In embodiments, based on the detected task or phase of the surgical procedure, the control algorithm may change the data recording rate, that is, to establish higher data sampling during dissection and suturing and to establish lower data sampling during instrument exchange and/or when the user is not engaged with the surgical console 30. The system 10 may determine disengagement of the user based on head tracking, eye tracking, hand contact with handle controllers 38a and 38b, or any other suitable means. The type of data logged may also be selected or modified by the control algorithm based on the task or phase of the surgical procedure. In embodiments, data or signals corresponding to arm torques may be logged when the control algorithm determines that the task is retraction, grasping force may be logged when the control algorithm determines that the task is fine manipulation, and no logging of signals when the control algorithm determines that the user is not engaged with the system 10.

In an aspect, the control algorithm determines the data logging rate and/or the rate of wireless transmission of data as a function of the movement speed of the robotic arm 40 and/or surgical instrument 50. In embodiments, when the control algorithm determines that the task calls for an increase in the speed of the robotic arm 40 and/or surgical instrument 50, the control algorithm will increase the data logging rate (and/or increase the rate of wireless transmission of data) and when the control algorithm determines that the task calls for a decrease in the speed of the robotic arm 40 and/or surgical instrument 50, the control algorithm will decrease the data logging rate (and/or reduce the rate of wireless transmission of data) to free up memory and bandwidth and reduce power consumption. Additionally, or alternatively, the control algorithm may synthesize or combine the data, prior to logging, to reduce bandwidth and the overall size of the data set.

The control algorithm determines when detected phases, tasks, or events are outside expected metrics or behaviors. In such instances where the control algorithm detects that a phase, task, or event is outside an expected metric or behavior, the control algorithm initiates the logging of high fidelity information to enable a more detailed post-analysis of the data. Certain procedural pre-defined or auto-identified actions may be recorded automatically by the control algorithm to catalog/document specific events of interest or clinical relevance, e.g., sample removal or bleed mitigation. The control algorithm may switch off data for certain system components when the control algorithm detects that such system components are out of view or when the task or phase does not involve them, thereby saving bandwidth, storage space, and reducing power consumption.

The control algorithm may also control user interface elements of the system 10 based on the detected phase or task. In embodiments, at a given predetermined phase or task, the control algorithm may cause a light (or other indicator) operably coupled to a robotic arm 40 to illuminate or a visual or audible indicator to activate on the display 32 of surgical console 30 or the display 23 of control tower 20 to indicate which surgical instrument 50 is likely to be exchanged next. As described above, icons to access and use tools or applications could become visible or more easily accessible on the display 32 of surgical console 30 or the display 23 of control tower 20 based on the detected phase or task. The control algorithm may additionally, or alternatively, cause display 32 or display 23 to display a list of surgical phases, steps, and tasks as a timeline for the specific procedure (e.g., as an overlay). The user can zoom in or navigate along the timeline to see next steps for the procedure, critical steps, or available tools used. In such a configuration, when the user navigates across the timeline to view details of previous steps or to view details of upcoming steps, the control algorithm can display a “home” button to return the user back to the current position along the timeline corresponding to the current step of the procedure.

In an aspect, the control algorithm can modify the color of the robotic arms 40 (e.g., via lights coupled to the robotic arms 40 or as images of the robotic arms 40 are display on display 23 of control tower 20 or any other display) or lights in the operating room or control tower 20 based on the phase or step being performed (e.g., blue for setup, yellow for dissection, purple for vessel sealing, orange for electrosurgery, green for suturing, etc.). This lets the operating room team know what is happening and link it to the surgical plan, which enables the operating room team to be ready for the next step or get the right tools and implantable devices ready. Additionally, the control algorithm may modify the function of the foot pedals 36 or buttons associated with the handle controllers 38a and 38b based on the detected phase or task.

The control algorithm may also adjust parameters associated with the user attention monitor based on the detected phase or step. In embodiments, if the current detected step is performed by an individual other than the user (e.g., by another member of the operating room staff), then the control algorithm can widen the attention monitor range and increase the scaling factor so that inadvertent motion of the handle controllers 38a and 38b has a small motion, or no motion, in the patient.

The control algorithm may adjust the volume of audible alarms and notifications based on the detected current surgical phase, step or task. In embodiments, lower volumes may be selected by the control algorithm or the control algorithm can reduce the volume, during delicate tasks when concentration is required, and higher volumes may be selected by the control algorithm or the control algorithm can increase the volume when there is more activity within the operating room and larger motions are used.

The control algorithm may enable live links or initiate remote communications to remote control systems or devices that enable feedback from mentors or other specialists, for example, to respond to inadvertent injury to a critical structure or organ that requires another consultant's guidance when such a phase or task is detected during a procedure. Additionally, or alternatively, for a user in a training program, certain phases may initiate mentorship connections or notify a mentor that a specific procedure step is coming and they need to be available.

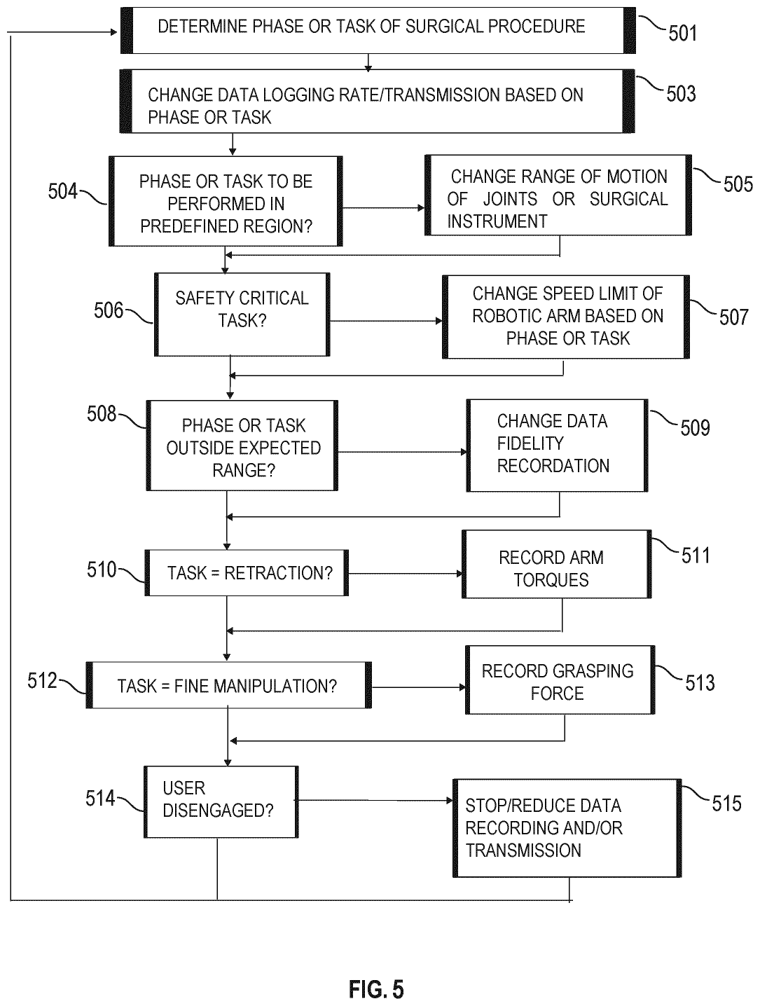

FIG. 5 illustrates a method for dynamically adjusting or controlling components of the surgical robotic system 10, and is illustrated as method 500. Method 500 may be an algorithm executed by a processor or controller of any component, or combination of components, of surgical robotic system 10. Although method 500 is illustrated and described as including specific steps, and in a specific order, method 500 may be carried out with fewer or more steps than described and/or in any order not specifically described.

Method 500 begins at step 501 where the phase, step, or task of the surgical procedure is determined. The phase, step, or commanded task is determined based on one or more sensors coupled to one or more components of the system 10, one or more sensors placed within the surgical setting, commands received from a user (e.g., commands received via any input devices such as handle controllers 38a and 38b or user interfaces), and/or inputs to and outputs form one or more other controllers of system 10.

In step 503, the data logging rate is adjusted based on the phase or task determined in step 501. In an aspect, adjusting the data logging rate includes modifying the type and/or amount of data stored within a components of system 10 or transmitted between components of system 10. For example, in an aspect, based on the phase or task determined in step 501, the type of data, or the amount of data, wirelessly transmitted between components of system 10 is adjusted. Depending on the phase or task determined, the amount (e.g., the size) of data wirelessly transmitted between components of system 10 is reduced, thereby utilizing less bandwidth. A reduction in bandwidth utilization enables more efficient and faster communications between components, reduced power consumption, reduced heat generation, faster processing speeds, etc.

In step 504, the system 10 determines whether the phase includes an operation that takes place in a predefined surgical area or whether the task is to be performed in the predefined surgical area. If the phase includes an operation that takes place in a predefined surgical area or whether the task is to be performed in the predefined surgical area, then in step 505, the range of motion of one or more joints of the robotic arm 40 or the range of motion of the surgical instrument 50 is changed (e.g., reduced).

In step 506, the system 10 determines whether the task is a safety critical task (e.g., a task that has been predefined as a safety critical task). If the task is a safety critical task, then in step 507, the speed limit of the robotic arm 40 is changed (e.g., reduced).

In step 508, the system 10 determines whether the phase or task is outside of an expected range (e.g., when detected phases, tasks, or events are outside expected metrics or behaviors). When it is determined that the phase or task is outside of an expected metrics range, then in step 509, the control algorithm initiates the logging of high fidelity information to enable a more detailed post-analysis of the data.

In step 510, the system 10 determines whether the task commands retraction of the surgical instrument 50 or robotic arm 40 (or whether the current phase includes retraction of the surgical instrument 50 or robotic arm 40). When it is determined that the current phase or task is retraction, then in step 511, the algorithm initiates the recordation of data corresponding to the torque values of the robotic arm 40.

In step 512, it is determined whether the phase or task includes fine manipulation (e.g., grasping, suturing, fine dissection) and if so, method 500 proceeds to step 513 where the algorithm initiates the recordation of data corresponding to grasping force of the surgical instrument 50.

In step 514, the system 10 determines whether the user is disengaged with components of the system 10 (e.g., surgical console 30). The system 10 may determine disengagement of the user based on head tracking, eye tracking, hand contact with handle controllers 38a and 38b, or any other suitable means. If the system 10 determines that the user is disengaged, then in step 515, the control algorithm reduces the logging of data signals, stops the logging of data signals, and/or reduces the wireless transmission of signals between components of the system 10, thereby utilizing less bandwidth.

While the disclosure contemplates and discloses applicability to wireless communication of data, it is contemplated and within the scope of the disclosure for the principles disclosed herein to apply equally to dedicated wired communications and or hybrid wired and wireless communications.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

1. A surgical robotic system, comprising:
a robotic arm including a surgical instrument coupled thereto;
a surgical console including:
a handle communicatively coupled to at least one of the robotic arm or the surgical instrument; and
a computer configured to:
determine a phase or a task of a surgical procedure based on at least one of sensor data or a user command to perform the task;
change a range of motion of one or more joints of the robotic arm or the surgical instrument based on the phase or the task of the surgical procedure;
change a speed limit of the robotic arm based on the phase or the task of the surgical procedure; and
change a rate of wireless transmission of data based on the phase or the task of the surgical procedure.

2. The surgical robotic system of claim 1, wherein the computer is further configured to change a speed limit of the robotic arm based on the phase or the task of the surgical procedure by increasing the speed limit of the robotic arm when the task is determined to be initial dissection.

3. The surgical robotic system of claim 1, wherein the computer is further configured to change a speed limit of the robotic arm based on the phase or the task of the surgical procedure by decreasing the speed limit when the task is determined to be a safety critical task.

4. The surgical robotic system of claim 1, wherein the computer is further configured to change a range of motion of one or more joints of the robotic arm based on the phase or the task of the surgical procedure by changing the motion scaling between the handle and the robotic arm.

5. The surgical robotic system of claim 1, wherein the computer is further configured to change a range of motion of the surgical instrument based on the phase or the task of the surgical procedure by reducing the range of motion of the surgical instrument when the task is to be performed in a predefined area.

6. The surgical robotic system of claim 1, wherein the computer is further configured to change an input mapping between the handle and the robotic arm and the surgical instrument based on the phase or the task of the surgical procedure.

7. The surgical robotic system of claim 6, wherein the computer is further configured to change the input mapping by amplifying rotation commands of the handle, remapping angles of the handle to change a start position of the handle, or changing control gains in the robotic arm.

8. The surgical robotic system of claim 1, wherein the computer is further configured to cause a display device to overlay measurement scaling over surgical images, display contrast media, display visual enhancements, or display at least one pre-operative image matching a current surgical view based on the phase or the task of the surgical procedure.

9. The surgical robotic system of claim 1, wherein the computer is further configured to change a rate of wireless transmission of data based on the phase or the task of the surgical procedure by increasing the rate of wireless transmission of data during dissection and suturing and reducing the rate of wireless transmission of data when a user is disengaged or when an instrument exchange is being performed.

10. The surgical robotic system of claim 1, wherein the computer is further configured to:
record arm torques in response to a determination by the computer that the task is retraction of the robotic arm or retraction of the surgical instrument;
record grasping force of the surgical instrument in response to a determination by the computer that the task is fine manipulation; and
stop record signals in response to a determination by the computer that a user is disengaged from the surgical console.

11. The surgical robotic system of claim 1, wherein the computer is further configured to change a data recording rate based on the phase of the surgical procedure and a speed of the surgical instrument by increasing the data recording rate when the speed of the surgical instrument is increased.

12. The surgical robotic system of claim 1, wherein the computer is further configured to:
determine if the phase or task is outside of an expected metrics range; and
record data with high fidelity information when it is determined that the phase or task is outside of the expected metrics range.

13. A surgical robotic system, comprising:
a surgical console including:
a handle communicatively coupled to at least one of a robotic arm or a surgical instrument; and
a computer configured to:
determine a phase or a task of a surgical procedure based on at least one of sensor data or a user command to perform the task;
determine whether the phase or task is to be performed in a predefined surgical area;
reduce a range of motion of the robotic arm or the surgical instrument when the task is to be performed in the predefined surgical area;
determine whether the phase or task is a safety critical task; and
decrease a speed limit of the robotic arm when the task is determined to be a safety critical task.

14. The surgical robotic system of claim 13, wherein the computer is further configured to:
determine if the phase or task is outside of an expected metrics range; and
record data with high fidelity information when it is determined that the phase or task is outside of the expected metrics range.

15. The surgical robotic system of claim 13, wherein the computer is further configured to:
determine whether the task is retraction; and
record arm torque data when it is determined that the task is retraction.

16. The surgical robotic system of claim 13, wherein the computer is further configured to:
determine whether the task is fine manipulation; and
record grasping force when it is determined that the task is fine manipulation.

17. A method for dynamic adjustment of a surgical robotic system including a robotic arm having a surgical instrument, the method comprising:
determining a phase or a task of a surgical procedure based on at least one of sensor data or a user command to perform the task;
determining whether the phase or task is outside of an expected metrics range;
determining whether the phase or task is to be performed in a predefined surgical area; and
recording data with high fidelity information when it is determined that the phase or task is outside of the expected metrics range or when the task is to be performed in the predefined surgical area.

18. The method of claim 17, further comprising:
recording arm torques when it is determined that the task is retraction of the robotic arm or retraction of the surgical instrument;
recording grasping force of the surgical instrument when it is determined that the task is fine manipulation; and
stopping the recording of signals when it is determined that a user is not engaged with the system.

19. The method of claim 17, further comprising adjusting a data recording or wireless transmission rate when a speed of the surgical instrument is modified.

20. The method of claim 17, further comprising changing a rate of wireless transmission of data based on the phase or the task of the surgical procedure by increasing the rate of wireless transmission of data during dissection and suturing and reducing the rate of wireless transmission of data when a user is disengaged or when an instrument exchange is being performed.
